(19) United States
(12) Patent Application Publication
(10) Pub. No.: US 2024/0285360 A1
(43) Pub. Date: Aug. 29, 2024

(54) SYSTEMS AND METHODS FOR SUPERVISED REMOTE IMAGING-GUIDED INTERVENTION

(71) Applicants: MASSACHUSETTS INSTITUTE OF TECHNOLOGY, Cambridge, MA (US); THE GENERAL HOSPITAL CORPORATION, Boston, MA (US)

(72) Inventors: Matthew R. JOHNSON, Medford, MA (US); Laura J. BRATTAIN, Lexington, MA (US); Brian A. TELFER, Lincoln, MA (US); Lars A. GJESTEBY, Maynard, MA (US); Joshua S. WERBLIN, Boston, MA (US); Nancy D. DELOSA, Westford, MA (US); Anthony E. SAMIR, Newton, MA (US); Theodore T. PIERCE, Auburndale, MA (US)

(21) Appl. No.: 18/499,179

(22) Filed: Oct. 31, 2023

Related U.S. Application Data
(60) Provisional application No. 63/420,900, filed on Oct. 31, 2022.

(51) Int. Cl. A61B 34/35; A61B 8/08; A61B 17/34; A61B 34/30

(52) U.S. Cl. CPC A61B 34/35; A61B 8/0841; A61B 17/3403; A61B 2017/3413; A61B 2034/303

(57) ABSTRACT

A method for remote intervention for a subject includes acquiring an image of a region of interest of the subject using an interventional device positioned on the subject and an image acquisition system. The region of interest includes a target structure and the subject is located at a first site. The method further includes analyzing the acquired image using an image analysis module to identify and label the target structure in the region of interest and transmitting the labelled image from the first site to a second site for expert review. The second site is remote from the first site. The method further includes receiving a command signal at the first site from the second site where the command signal is generated based on the expert review of the labelled image and configured to control an action of the interventional device. In some embodiments, the method may further include analyzing the acquired image to determine a pathway to the vessel that avoids critical structures.

### CROSS-REFERENCE TO RELATED APPLICATIONS

This application is based on, claims priority to, and incorporates herein by reference in its entirety U.S. Ser. No. 63/420,900 filed Oct. 31, 2022, and entitled “Systems and Methods for Supervised Remote Ultrasound-Guided Intervention.”

### STATEMENT REGARDING FEDERALLY SPONSORED RESEARCH

This invention was made with government support under FA8702-15-D-00001 awarded by the U.S. Army and Defense Health Agency. The government has certain rights in the invention.

### BACKGROUND

There is increasing interest and investment in facilitating the home-based healthcare model, wherein health services are largely provided via remote interactions (i.e., telehealth). These initiatives, generally named “Hospital at Home,” are ramping up at major health care systems across the U.S. and aim to improve care and reduce costs by reducing hospital stays by providing improved, extended care at home.

### SUMMARY OF THE DISCLOSURE

In accordance with an embodiment, a method for supervised remote intervention for a subject includes acquiring an image of a region of interest of the subject using an interventional device positioned on the subject and an image acquisition system. The region of interest includes a target structure and the subject is located at a first site. The method further includes analyzing the acquired image using a machine learning network to identify and label the target structure in the region of interest and transmitting the labelled image from the first site to a second site for expert review. The second site is remote from the first site. The method further includes receiving a command signal at the first site from the second site where the command signal is generated based on the labelled image and configured to control an action of the interventional device.

In some embodiments, analyzing the acquired image further includes analyzing the acquired image to detect critical structures that a needle should avoid and computing a pathway from a surface of the subject such that the needle avoids the critical structures and intersects with the target structure. In some embodiments, the method further includes causing the deployment of a needle of the interventional device based on the command signal. In some embodiments, the method further includes enabling the interventional device for deployment based on the command signal and causing the deployment of a needle of the interventional device. In some embodiments, the image analysis module is implemented as a machine learning network. In some embodiments, the interventional device is a vascular access device configured for drawing blood. In some embodiments, the interventional device is a vascular access device configured for intravenous medicine delivery. In some embodiments, the interventional device includes an ultrasound transducer and the image acquisition system is an ultrasound system. In some embodiments, the interventional device includes an optical image sensor and the image acquisition system is an optical imaging system. In some embodiments, transmitting the labelled image from the first site to a second site for expert review includes transmitting the labelled image from the first site to the second site over a communication network. In some embodiments, the interventional is a vascular access device and the target structure is a target vessel, and analyzing the acquired image using an image analysis module to identify and label a target structure in the region of interest includes determining one or more of a location of the target vessel, a centroid depth of the target vessel, and a diameter of the target vessel. In some embodiments, the method further includes determining if the target vessel is appropriate for needle insertion based on the determined diameter of the target vessel. In some embodiments, the interventional device is configured to be positioned around an arm of the subject and the interventional device can include a cuff configured to be positioned around an arm of the subject. In some embodiments, the method further includes monitoring the interventional device based on images acquired using the interventional device and the image acquisition system to determine a change in position of the vascular access device.

In accordance with another embodiment, a system for remote intervention for a subject, includes an interventional device positioned on the subject. The interventional device includes a an image sensor, a needle, a robotic assembly comprising a needle positioning system configured to automatically adjust a position of the needle with respect to the image sensor to align the needle with a target structure in a region of interest of the subject. The system further includes an image acquisition system coupled to the image sensor of the interventional device, and an image analysis module coupled to the interventional device and the image acquisition system. The image analysis module is configured to analyze an image of the region of interest of the subject to identify and label the target structure, and to determine a pathway to the vessel that avoids critical structures. The image of the region of interest is acquired using the image sensor and image acquisition system.

In some embodiments, the image analysis module is a machine learning network. In some embodiments, the needle positioning system is further configured to automatically adjust the position of the needle to align the needle with a target insertion point for the target structure and to avoid critical structures. In some embodiments, the interventional device is a vascular access device and further includes a cuff configured to be positioned around an arm of the subject. In some embodiments, the interventional device is a vascular access device configured for drawing blood and further includes one or more vials. In some embodiments, the interventional device is a vascular access device configured for intravenous medicine delivery. In some embodiments, the image sensor is a transducer array and the image acquisition system is an ultrasound system. In some embodiments, the image sensor is an optical image sensor and the image acquisition is an optical imaging system. In some embodiments, the interventional service is a vascular access device, the target structure is a target vessel and the image analysis module is further configured to determine one or more of a location of the target vessel, a centroid depth of the target vessel, and a diameter of the target vessel. In some embodiments, the interventional device is a vascular access device configured to be positioned around an arm of the subject and to constrict around the arm of the subject to increase the diameter of the target vessel.

In accordance with another embodiment, a method for remote intervention for a subject includes acquiring an image of a region of interest including a target structure of the subject using an interventional device positioned on the subject and an image acquisition system, analyzing the acquired image using an image analysis module to identify and label a target structure in the region of interest and generating a command signal, using the image analysis module, the command signal generated based on the labelled image and configured to control an action of the interventional device.

### BRIEF DESCRIPTION OF THE DRAWINGS

The present invention will hereafter be described with reference to the accompanying drawings, wherein like reference numerals denote like elements.

FIG. 1 is a block diagram of a system for supervised remote interventional procedures in accordance with an embodiment;

FIG. 2 illustrates a method for supervised remote interventional procedures in accordance with an embodiment;

FIG. 3 illustrates an example supervised remote phlebotomy system in accordance with an embodiment;

FIG. 4A illustrates a top view of an example remote vascular access device in accordance with an embodiment;

FIG. 4B illustrates a back view and a side view of the example remote vascular access device of FIG. 4A in accordance with an embodiment;

FIG. 5 is a block diagram of an example computer system in accordance with an embodiment; and

FIG. 6 is a schematic diagram of an example ultrasound system in accordance with an embodiment.

### DETAILED DESCRIPTION

The present disclosure describes systems and methods for supervised remote imaging-guided intervention. In some embodiments, the described systems and methods can extend the capability of at-home services (or point-of-care services located in other non-hospital or non-laboratory settings, for example, a pharmacy clinic) to include supervised remote intervention for applications including, but not limited to, remote vascular access (e.g., phlebotomy, intravenous delivery of medications, or IV placement), remote injection into muscles, remote injection of medicine into body cavities, and remote injection or placement of interventional devices into organs such as the liver, brain, and kidneys. In some embodiments, the described systems and methods allow for remote expert supervision for a remote interventional procedure for a subject. In some embodiments, the described systems and methods can allow for remote supervision of access to the arterial system of a subject for the purposes of performing a remotely controlled endovascular procedure (or intervention). Accordingly, in some embodiments, home-based patients and caregivers (e.g., family members) can use the described systems and methods to, for example, sample blood or deliver intravenous medications.

For the purposes of this disclosure and accompanying claims, the term “real time” or related terms are used to refer to and define a real-time performance of a system, which is understood as performance that is subject to operational deadlines from a given event to a system's response to that event. For example, a real-time extraction of data and/or displaying of such data based on acquired image data may be one triggered and/or executed simultaneously with and without interruption of a signal-acquisition procedure.

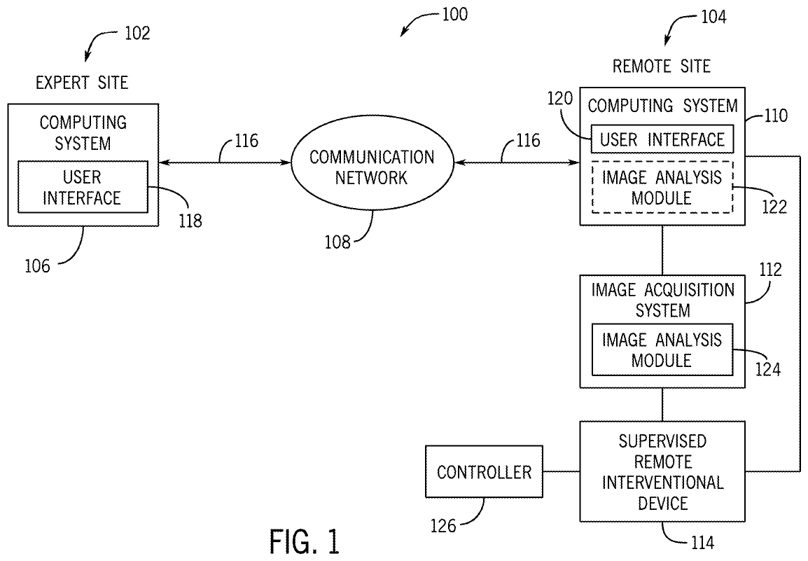

FIG. 1 is a block diagram of a system for supervised remote interventional procedures in accordance with an embodiment. The system 100 can include a computing system 106 located at an expert site 102 (e.g., an office, a hospital), and a computing system 110, image acquisition system 112, and supervised remote interventional device 114 at a remote site 104 (e.g., a subject's home or other non-hospital or non-laboratory setting). As used herein, the remote site 104 may be the location of the subject and, in some embodiments, a caregiver, and the expert site 102 may be the location of an individual with expertise (i.e., an expert) in image interpretation and interventional procedures (e.g., for vascular access) such as, for example, a doctor, phlebotomist, nurse, etc.). In some embodiments, the computer system 106 and the computer system 110 may be any general-purpose computing system or device, such as a personal computer, workstation, cellular phone, smartphone, laptop, tablet or the like. As such, computer system 106 and computer system 110 may include any suitable hardware and components capable of carrying out a variety of processing and control tasks in accordance with aspects of the present disclosure. For example, the computer system 106 and the computer system 110 may include a programmable processor or combination of programmable processors, such as central processing units (CPUs), graphics processing units (GPUs), and the like. In some embodiments, the computer system 106 and the computer system 110 may be configured to execute instructions stored on in a non-transitory computer readable media. In some embodiments, the computer system 106 can include a user interface 118 and the computing system 110 can include a user interface 120. User interface 118 and user interface 120 can include, for example, a display and one or more input devices (e.g., a keyboard, a mouse, a touchscreen).

The computing system 106 at the expert site 102 and the computing system 110 at the remote site 104 may be in communication over a communication network 108. In an embodiment, the expert site 102 and the remote site 104 are located away from one another, for example, different locations in the same building, different buildings in the same city, different cities, or other different locations where an expert at the expert site 102 does not have physical access to the subject or the remote vascular access device 114. In some embodiments, the computing system 106 and the computing system 110 may be configured to include telepresence capabilities (e.g., software applications, a video camera, monitors, speakers and microphone(s)) configured to provide audio and video communication (e.g., telepresence, teleconference, video conference) between the expert at the expert site 102 and the patient and, in some embodiments, the caregiver, at the remote site 104. In some embodiments, the expert at the expert site 102 may utilize the computing system 102 to communicate with the patient (and caregiver) via the computing system 110 at the remote site 104 and oversee and/or effect the actions of a supervised remote interventional device 114 to, for example, draw blood from the subject, deliver intravenous medication to the subject, place an IV in the subject, or remotely place an arterial access needle, sheath or wire in the subject. For example, a video conference may be established between the computing system 106 at the expert site 102 and the computing system 110 at the remote site 104 so that the expert at the expert site 102 may view the patient and, in some embodiments, the caregiver, at the remote site 104, and the expert at the expert site 102 and the patient/caregiver at the remote site 104 may communicate via audio and video.

In some embodiments, communication network 108 can be any suitable communication network or combination of communication networks. For example, communication network 108 can include a Wi-Fi network (which can include one or more wireless routers, one or more switches, etc.), a peer-to-peer network (e.g., a Bluetooth network), a cellular network (e.g., a 3G network, a 4G network, a 5G network, etc., complying with any suitable standard, such as CDMA, GSM, LTE, LTE Advanced, WiMAX, etc.), a wired network, and so on. In some embodiments, communication network 108 can be a local area network, a wide area network, a public network (e.g., the Internet), a private or semi-private network (e.g., a corporate or university intranet), any other suitable type of network, or any suitable combination of networks. Communications links 116 shown in FIG. 1 can each be any suitable communications link or combination of communications links, such as wired links, fiber optic links, Wi-Fi links, Bluetooth links, cellular links, and so on.

At the remote site 104, the computing system 110 may be coupled to and in communication with an image acquisition system 112 and the supervised remote interventional device 114. The remote interventional device 114 may be configured for various types of remote interventions that include deploying a needle (e.g., for an injection) in a target anatomy or target structure of the subject. For example, in some embodiments, the remote interventional device 114 may be a remove vascular access device (e.g., for phlebotomy, intravenous delivery of medications, or IV placement), a remote interventional device for injection (e.g., medicines) into muscles, a remote interventional device for injection of medication into body cavities, or a remote interventional device or for injection or placement of another interventional device into organs such as, for example, the liver, brain, and kidneys. In some embodiments, the target structure of the subject can include, for example, an artery, a vein, a femoral artery, a femoral vein, a jugular vein, a peripheral vein, a subclavian vein, an airway, a lumen, a luminal organ, a body cavity, a fluid filled anatomic space, a location requiring biopsy, a breast, a kidney, a lymph node, a spinal canal, a location requiring nerve block, a peritoneal space, or a pleural space. While the following description of FIG. 1 may refer to a remote vascular access device for the remote interventional device, it should be understood that other types of remote interventional devices configured to target other structures (or anatomy) of the subject than the vessels, may be utilized in the system 100.

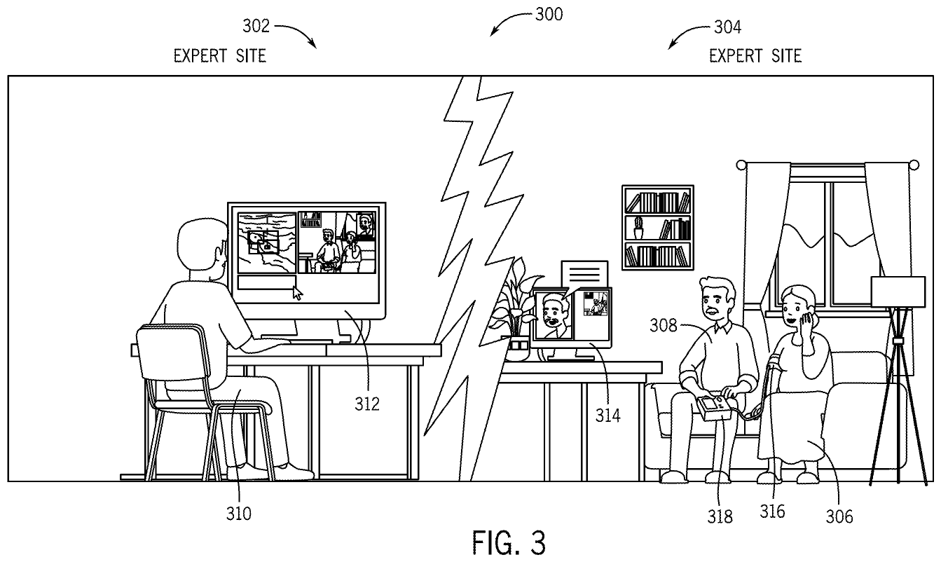

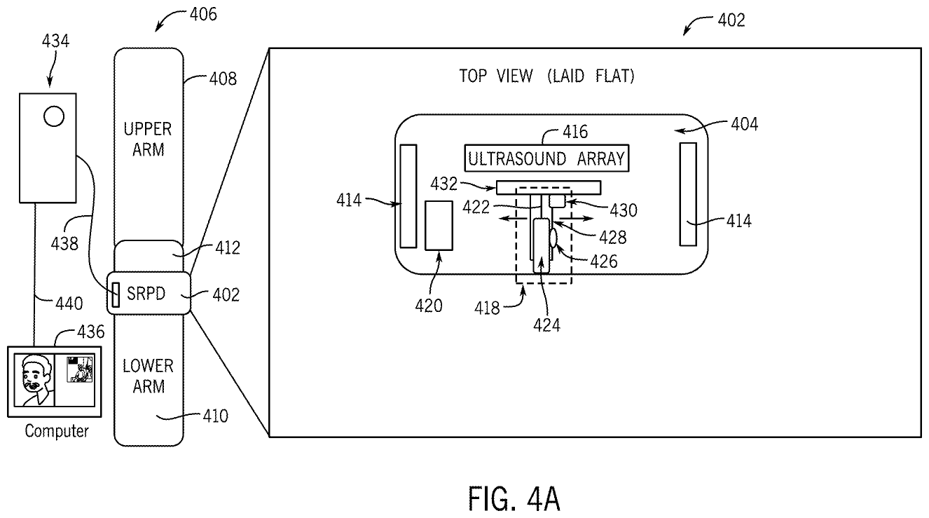

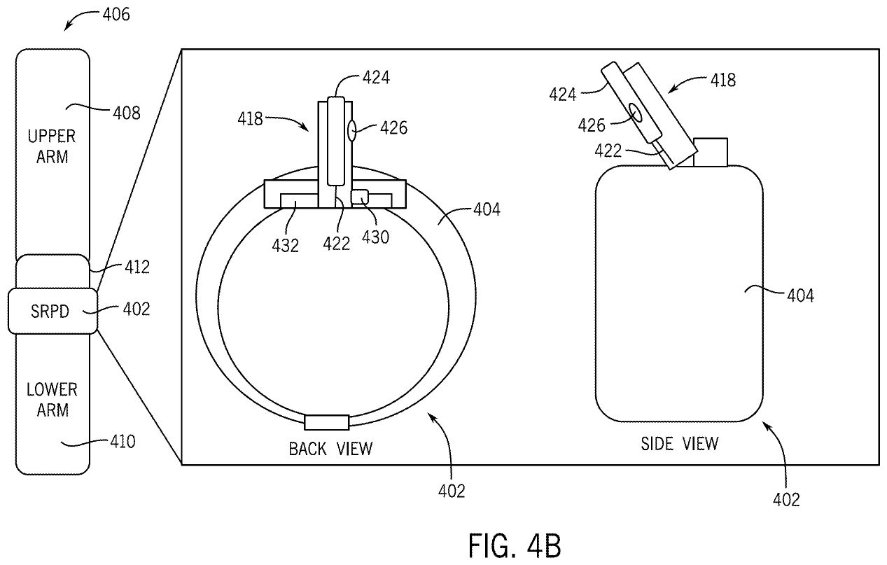

In some embodiments, the remote interventional device 114 (e.g., a remote vascular access device) may be configured as an “arm-band” or “cuff” type robotic assembly that may be positioned on or attached to a subject's arm between the shoulder and wrist (e.g., either proximal or distal to the subject's elbow) to insert a needle into a target vessel (or other target structure) of the subject to, for example, draw blood or deliver intravenous medicine. In some embodiments, the remote interventional device 114 may be configure to be positioned on other areas of the subject (e.g. other body parts). In some embodiments, the remote interventional device 114 can include, for example, image sensor(s) (e.g., an ultrasound transducer array), a needle, vial(s), a robotic assembly or system for performing needle positioning and insertion, and a needle actuation controller. The image sensor(s) may be coupled to the image acquisition system 112 to acquire and generate images of a region of interest of the subject (e.g., the area proximal or distal to the elbow) to identify a target structure (e.g., a target vessel) for needle insertion. In some embodiments, the image sensor(s) may be an ultrasound transducer incorporated into the “arm band” assembly and the image acquisition system may be an ultrasound system. While the following description will refer to embodiments utilizing ultrasound technology it should be understood that other imaging technologies may be utilized such as, for example, video or optical imaging. Accordingly, the image sensor(s) can be the appropriate image sensor(s) for the imaging technology used, for example, cameras for video imaging or optical image sensor(s) for optical imaging. In addition, the image acquisition system 112 can be the appropriate imaging system for the implemented imaging technology. In some embodiments, the remote interventional device 114 may be positioned on or attached to a subject's arm (or other area or region of the subject) such that the target structure (e.g. a target vessel) is within the field of view of the image sensor(s). The needle(s) provided in the remote interventional device 114 may be the appropriate size for the particular application of the remote interventional device 114 (e.g., drawing blood, intravenous medicine delivery). In some embodiments, for drawing blood, the robotic assembly may be configured to actuate a needle, for example, to cause deployment of the needle to insert the needle in the target vessel and to fill one or more vial(s) in the remote interventional device with blood. In some embodiments, the robotic assembly may be configured to actuate a needle, for example, to cause deployment of the needle to insert the needle in the target vessel and to deliver medicine from one or more vial(s) to the subject. As mentioned above, the supervised remote interventional device 114 may also include a needle actuation controller. In some embodiments, the needle actuation controller may be incorporated in the “arm band” or “cuff” assembly and, in some embodiments, the needle actuation controller may be a controller 126 coupled to the supervised remote interventional device 114 via, for example, a cable or wire. For example, the controller 126 may be incorporated in a hand held device (e.g., controller 318 shown in FIG. 3 or controller 434 shown in FIG. 4A). In some embodiments, the remote interventional device 114 or the controller 126 may include a user input (e.g., a button) the subject or caregiver at the remote site 104 can use to initiate needle deployment.

An image analysis module may also be provided that can be configured to analyze the images acquired by the supervised remote interventional device 114 to identify the target structure (e.g., a target vessel) and to segment or label the acquired images and the target structure (e.g. a target vessel). For example, in some embodiments, an image analysis module 124 may be implemented in the image acquisition system 112 at the remote site 104 and, in some embodiments, an image analysis module 122 may optionally be implemented on the computer system 110 at the remote site 104. In some embodiments, the image analysis module 122, 124 may be implemented as a trained machine learning network (e.g., a neural network), an AI routine, or image analysis algorithm. In some embodiments, image analysis module 122, 124 may be configured to determine a location of the structure (e.g., a target vessel) and various characteristics of the structure, for example, for a vessel characteristics such as, vessel centroid depth, diameter, location along the image sensor (e.g., an ultrasound array). Segmentation of the structure (e.g., a target vessel) may be based on machine learning of morphological and spatial information in the images of the region of interest and the target structure (e.g., ultrasound images). In some embodiments, a neural network may be trained to learn features at multiple spatial and temporal scales. In one example, vessels of interest may be distinguished based on shape and/or appearance of the vessel wall, shape and/or appearance of surrounding tissues, and the like. Characteristics such as vessel diameter, etc., may be used to determine if a vessel is appropriate for needle insertion. As mentioned, in some embodiments, the image analysis module 122, 124 for analyzing the acquired image(s) to identify the target structure and to segment or label the acquired image(s) and the target structure may be implemented as an AI routine or an image analysis algorithm (or module). Advantageously, the machine leaning network, AI algorithm or image analysis algorithm can be implemented at the remote site 104 and can therefore be applied to images acquired locally from the subject. In some embodiments, an insertion point may be determined based on the determined location of the target structure and calculating a depth and a pathway for a needle of the remote interventional device 114 from the surface of the subject to the target structure. In addition, the image analysis module 122, 124 may also be configured to analyze an acquired image or images to detect critical structures that a needle should avoid and to compute a pathway, for example, from a surface (e.g., skin) of the subject to the target structure such that the needle avoids the critical structures and intersects with the target structure.

The labeled (or annotated) image of the region of interest and the target structure (e.g., a target vessel) generated by the image analysis module 122, 124 may be transmitted to the computing system 106 at the expert site 102 and displayed to the expert (e.g., a display of the user interface 118). The expert can advantageously review the labeled image and determine, for example, if the needle of the remote interventional device 114 is positioned correctly for needle insertion into the target structure (e.g. a target vessel) of the subject. If the needle is positioned correctly, the expert may provide a user input to the computing system 106 (e.g., via user interface 118) to generate a command signal. In some embodiments, the command signal maybe configured to enable (or “arm”) a needle insertion function on the remote interventional device 114. In some embodiments, the command signal may be configured to activate the remote interventional device 114 and cause deployment of the needle to, for example, insert the needle into the target structure. The command signal may be transmitted to the computing system 110 and the remote interventional device 114 at the remote site 104. In some embodiments where the command signal is configured to enable the needle injection function, the expert may also provide instructions to the subject or caregiver at the remote site 104 to initiate the needle deployment, for example, by pressing a button on the remote interventional device 114 or controller 126. The robotic assembly (e.g., a needle insertion system and/or needle actuation controller) may then be used to automatically deploy the needle to insert the needle into the target structure (e.g., a target vessel). If the needle of the remote interventional device 114 is not positioned correctly, the expert may provide instructions to the subject or caregiver to adjust the position of the remote interventional device 114 on the subject (e.g., on the subject's arm or other area). The remote interventional device 114 and image acquisition system 112 may then be used to acquire images of the region of interest at the new position and the images may be processed (e.g., using the image analysis module 122, 124) to identify and label the target structure. The expert may then review the labeled image for the new position and determine whether to enable the needle injection function of the remote interventional device 114 or cause the deployment of the needle of the remote interventional device 114 (i.e., determine whether the needle is positioned correctly). In some embodiments, the image analysis module 122, 124 and annotated images may be unsupervised, namely, review and verification by an expert may not be required. In some embodiments, the image analysis module 122, 124 and annotated images may be supervised by a person with less expertise than a specialist. In some embodiments, the image analysis module 122, 124, rather than an individual, may be used (and configured) to automatically determine if the needle of the remote interventional device 114 is positioned correctly for needle insertion into the target structure of the subject. If the needle is positioned correctly, the image analysis module 122, 124 may generate a command signal, for example, to enable (or “arm”) a needle insertion function on the remote interventional device 114 or to cause deployment of the needle of the remote interventional device 114 to, for example, insert the needle into the target structure.

In some embodiments, the image analysis module 122, 124, image sensors in the supervised remote interventional device 114, and image acquisition system 112 may be configured to monitor the position of the remote interventional device 114 (e.g., the needle) in real time and determine if the remote interventional device 114 moves or changes position, for example, during the time the a labeled image is transmitted to the expert site 102 from the remote site 104 (and is reviewed by the expert) and before receiving a command signal at the remote site 104 from the expert site 102, or between receiving the command signal at the remote site 104 and the user initiating the deployment of the needle. This feature may be advantageous for patient safety and may be used to mitigate, for example, communication network time delays and motion artifacts. For example, there may be a delay in communication of data and images over the communication network 108 between the remote site 104 and the expert site 102. During the communications delay, the subject may move causing the position of the remote interventional device 114 (e.g., the needle) to shift. By monitoring the position of the remote interventional device 114 in real time, the system and method can disable the needle injection function until it is determined whether the new position of the remote interventional device 114 is appropriate for needle injection or if the remote interventional device 114 should be repositioned on the subject's arm. In some embodiments, the expert at the expert site 102 may wish to select a different target than identified by the image analysis module. In some embodiments, if communications have been lost between the expert site and the remote site, various elements of the system at the remote site may be configured to disable the needle injection function.

In some embodiments, the robotic assembly (or system for performing needle positioning and insertion) and a controller may be configured to allow for adjustment of the positioning of the needle in the remote interventional device 114. In some embodiments, the robotic assembly of the remote interventional device 114 may include mechanisms to automatically adjust an angle of the needle relative to a surface of the subject. In some embodiments, the robotic assembly may advantageously be configured to provide an additional degree of freedom for the needle which can advantageously allow automatic fine tuning of the position of the needle with respect to the target structure (e.g., a target vessel) and an appropriate insertion point. For example in some embodiments, the robotic assembly may be configured to include mechanism (e.g., a needle translation track) that allow a translational position of the needle to be automatically adjusted along the image sensor (e.g., an ultrasound array). The additional degree of freedom can act to slide (e.g., along a needle translation track) the needle across the image sensor for a “fine-positioning” step prior to needle insertion. This feature can be advantageous by enabling a user (e.g., a subject or caregiver) with limited dexterity to use the remote interventional device. As a result, the user only needs to position the remote interventional device such that the target structure is within the field of view of the image sensor (e.g., for an ultrasound transducer, within approximately 4 cm).

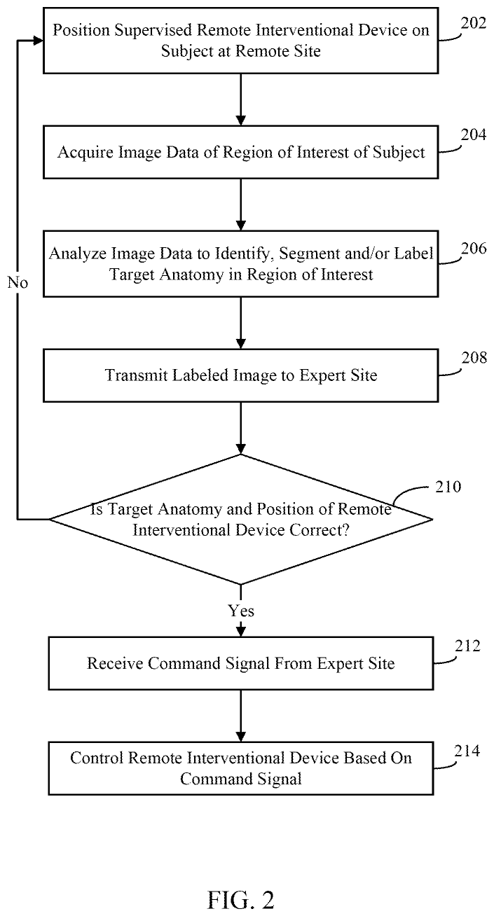

FIG. 2 illustrates a method for supervised remote vascular access in accordance with an embodiment. The process illustrated in FIG. 2 is described below as being carried out by the system 100 for supervised remote vascular access as illustrated in FIG. 1. Although the blocks of the process are illustrated in a particular order, in some embodiments, one or more blocks may be executed in a different order than illustrated in FIG. 2, or may be bypassed. While the following description of FIG. 1 refers to a remote vascular access device as the remote interventional device and a target vessel as the target structure, it should be understood that other types of remote interventional devices and target structures (or anatomy) of the subject, for example, as discussed above, may be utilized in the process of FIG. 2.

At block 202, a remote interventional device 114 (e.g., a remote vascular access device) may be positioned on a subject at a remote site 104. For example the subject or a caregiver for the subject may attach the remote vascular access device to a subject's arm so that an image sensor in the remote vascular access device may acquire an image of a region of interest. In some embodiments, for needle insertion (e.g., for blood collection or intravenous drug delivery) the remote vascular access device may be configured as an “arm band” or “cuff” that may be positioned on the subject's arm between the shoulder and wrist (e.g., either proximal or distal to the elbow of the subject). In some embodiments, the remote vascular access device may be positioned on or attached to a subject's arm such that a target structure (e.g., a target vessel) is within the field of view of image sensor(s) of the remote vascular access device 114. At block 204, image data (or image(s)) of the region of interest may be acquired using, for example, the image sensor(s) in the remote vascular access device and an image acquisition system 112 coupled to the image sensor(s). In some embodiments, the image sensor can be an ultrasound transducer incorporated in the remote vascular access device and the image acquisition system 112 can be an ultrasound system (e.g., a portable ultrasound system). As mentioned above, in some embodiments, other imaging technologies may be utilized such as, for example, video or optical imaging. Accordingly, the image sensor(s) and the image acquisition system 112 may be the appropriate image sensor(s) and imaging system for the implemented imaging technology.

At block 206, the acquired image data (or image(s)) may be analyzed to identify a target structure (e.g., a target vessel) in the region of interest and to segment and/or label the target structure (e.g., a target vessel) in the region of interest. In some embodiments, as discussed above with respect to FIG. 1, an image analysis module 122, 124 (e.g., a trained machine learning network (e.g., a neural network), an AI routine, or image analysis algorithm) may be used to analyze the acquired image data to identify and label the target vessel. In some embodiments, the image analysis module 122, 124 may be configured to determine a location of the target vessel and various vessel characteristics such as, for example, vessel centroid depth, diameter, location along the image sensor (e.g., an ultrasound array). In some embodiments, an insertion point may also be determined (e.g., using an image analysis module 122, 124) based on the determined location of the target vessel and calculating a depth and a pathway for a needle of the remote vascular access device 114 from the surface of the subject to the target vessel. In addition, in some embodiments, an acquired image or images can be analyzed (e.g., using an image analysis module, 122, 124) to detect critical structures that a needle should avoid and to compute a pathway, for example, from a surface (e.g., skin) of the subject to the target vessel such that the needle avoids the critical structures and intersects with the target vessel.

At block 208, the labeled or annotated image(s) may be transmitted from the remote site 104 (or location of the subject) to an expert site 102 for review by an expert (e.g., a doctor, phlebotomist, nurse, etc.). In some embodiments, the labeled image may be transmitted from a computing system 110 at the remote site 104 to a computing system 106 at the expert site 102 via a communication network 108. In some embodiments, the labeled image may be displayed to the expert, for example, using a display (e.g., of a user interface 118) of the computing system 106 at the expert site 102. The expert may then review the labeled image to determine if a needle of the remote vascular access device is correctly positioned to proceed with needle insertion in the target structure (e.g., a target vessel). At block 210, if the placement of the needle is not correct, the process may return to block 202 and the subject or the caregiver may adjust the position of the remote vascular access device and, therefore, the position or placement of the needle of the remote vascular access device with respect to the target vessel. Image acquisition and analysis at blocks 204 and 206 may then be performed for the new position of the needle (and remote vascular access device). In some embodiments, the annotated images may be unsupervised, namely, review and verification by an expert may not be required. In some embodiments, the annotated images may be supervised by a person with less expertise than a specialist. In some embodiments, an image analysis module 122, 124, rather than an individual, may be used to determine if the needle of the remote vascular access device is positioned correctly for needle insertion into the target vessel of the subject and to generate a command signal.

If, at block 210, the needle is in the correct position, a command signal (e.g., generated by the computing system 106) may be received at the remote site 104 from the expert site 102. In some embodiments, the command signal may be configured to enable (or “arm”) a needle insertion function of the remote vascular access device. In some embodiments, the command signal may be configured to activate the remote vascular access device and cause deployment of the needle to, for example, insert the needle into the target structure (e.g., a target vessel). For example, the expert at the expert site 102 may provide a user input to the computing system 106 (e.g., via a user interface 118) that generates a command signal and the command signal may then be transmitted to the computing system 110 and remote vascular access device at the remote site 104. At block 214, the remote interventional device 114, for example, a remote vascular access device, may be controlled based on the received command signal. In some embodiments, deployment of the needle for insertion in a target structure (e.g., a target vessel) may be initiated. For example, once the needle function has been enabled based on the command signal the subject or caregiver may press a button on the remote vascular access device (or controller 126) to initiate needle deployment (or actuation). In another example, the command signal may cause the deployment of the needle in the remote vascular access device to, for example, inject the needle into a target vessel.

As mentioned above, in some embodiments, the remote interventional device (e.g., a remote vascular access device) may be configured to draw blood from a subject. FIG. 3 illustrates an example supervised remote phlebotomy system in accordance with an embodiment. In FIG. 3, a subject (e.g., a patient) 306 and caregiver 308 located at a remote site 304 (e.g., the subject's home or other non-hospital or non-laboratory setting) and an expert 310 (e.g., a doctor, phlebotomist, nurse, etc.) located at an expert site 302 (e.g., an office, a hospital, home workstation, etc.) may communicate via, for example, video conference over a communication network (e.g., communication network 108 shown in FIG. 1). In some embodiments, the expert 310 may supervise the subject 306 and caregiver 308 while performing a blood draw for the subject 306. The following discussion of FIG. 3 describes an example workflow for supervised remote phlebotomy. The workflow and enabling software and devices can advantageously provide for home-based (e.g., Hospital at Home) point of care for blood collection. In some embodiments, a physician may determine that a subject 306 (e.g., a patient) requires a blood sample be taken (e.g., for analysis). For example, the physician may determine that a blood sample is required for a subject 306 in an office visit or in a visit via video conference. If a blood sample is required, the physician may order a supervised remote phlebotomy “kit” to be sent to the subject 306 at home (e.g., remote site 304). In some embodiments, the remote phlebotomy kit may include a remote vascular access device 316 in the form of a remote phlebotomy device that can include empty pre-loaded blood vials, a controller 318 (e.g., a handheld controller) for the remote phlebotomy device 316, a trained machine learning network for image analysis, a portable image acquisition system (e.g., a portable ultrasound system), use instructions, and an appropriate sample return container (e.g., with pre-paid shipping). Once the remote phlebotomy kit is received, a caregiver 308 for the subject 306 may open the kit and use a computer system 314 at the remote site 304 to connect to a computer system 312 of the expert 310 at the expert site 302, for example, via a video conference. In some embodiments, the subject 306 may perform the remote blood collection themselves rather than with the assistance of a caregiver 308.

The expert 310 (e.g., a doctor, nurse, phlebotomist, etc.) can walk the subject 306 or caregiver 308 through a setup process, for example, including sterilization, topical anesthetic (if needed), and gross placement of the remote phlebotomy device 316 on the subject 306 (e.g., on the subject's arm). In some embodiments, as discussed above with respect to FIG. 1 and discussed further below with respect to FIGS. 4A and 4B, the remote phlebotomy device 316 may be configured to perform “fine tuning” of the position of the needle in the remote phlebotomy device 316 to precisely position the needle relative to the target vessel. The expert 310 may review (e.g., on computer system 312 at the expert site 302) a labeled image of the region of interest and the target vessel that is generated using image sensors in the remote phlebotomy device 316, the portable image acquisition system (e.g., a portable ultrasound system/device) and the trained machine learning network. The labeled image (or images) may be transmitted to the expert site 302 from the remote site 304 over a communication network. The expert 310 may review the labeled image to determine if whether to proceed to draw blood from the subject 306 based on the current position of the needle of the remote phlebotomy device 316. If the expert 310 determines it is acceptable to proceed, the expert 310 can remotely enable a needle injection function of the remote phlebotomy device 316 and instruct the subject 306 or caregiver 308 to, for example, press a button on the remote phlebotomy device 316 or the controller 318 to deploy (or actuate) the needle in the remote phlebotomy device 316 and initiate the blood draw. When the blood draw is initiated, the remote phlebotomy device 316 can deploy the needle to inject the needle into the target vessel and draw blood into the pre-loaded vial until the vial is filled to a predetermined amount. The remote phlebotomy device 316 may then retract the needle to withdraw the needle from the subject. In some embodiments, the pre-labeled vial containing the subject's blood may be ejected or removed from the remote phlebotomy device 316. The subject 306 or caregiver 308 may then be instructed to remove the remote phlebotomy device 316 from the subject's arm. The expert 310 or a designee of the expert may then provide the subject 306 or caregiver 308 instructions on placing a bandage and the expert 310 (or a designee of the expert) may also monitor the subject 306 for a brief observation period. Once the procedure is completed, the subject 306 or caregiver 308 may place the blood sample in the blood vial(s) in the sample return container with, for example, pre-paid shipping to return the blood sample and devices to a blood analysis laboratory. The received blood sample may be analyzed and the blood analysis lab may post the lab results to the subject's medical file. In some embodiments, the remote phlebotomy device 316 and portable ultrasound device may also be placed in the same or different container and returned to the medical laboratory or other appropriate entity.

FIG. 4A illustrates a top view of an example remote vascular access device in accordance with an embodiment and FIG. 4B illustrates a back view and a side view of the example remote vascular access device of FIG. 4A in accordance with an embodiment. The example remote vascular access device in FIGS. 4A and 4B is configured as a supervised remote phlebotomy device (SRPD). As mentioned above, in some embodiments, the remote vascular access device may be configured for other applications such as intravenous delivery of medicine and placement of an IV. In some embodiments, the remote phlebotomy device 402 may be configured as an “arm band” or “cuff” 404 (e.g., similar to a blood pressure cuff) that may be positioned around an arm of the subject. As shown in FIGS. 4A and 4B, for drawing blood, the remote phlebotomy device 402 may be positioned around the subject's arm 406 between the shoulder and wrist, either proximal or distal to the elbow 412. For example, in some embodiments, the remote phlebotomy device 402 may be positioned around the subject's lower arm 410 distal to the elbow 412 or around the subject's upper arm 408 proximal to the elbow 412. In FIGS. 4A and 4B, the remote phlebotomy device 402 is illustrated as positioned around the subject's lower arm 410 distal to the elbow 412. In some embodiments, the remote phlebotomy device 402 may be coupled to and in communication with a controller 434 (e.g., controller 126 shown in FIG. 1) via a connector 438 (e.g., a cable). The remote phlebotomy deice 402 and/or the controller 434 can be in communication with a computing system 434 (e.g., computing system 110 shown in FIG. 1) at the location of the subject (i.e., a remote site) via a communication link 440 (e.g., a wire or wireless communication link).

In FIG. 4A, a top view of the remote phlebotomy device 402 with the cuff 404 laid flat is shown. The cuff 404 may include an attachment mechanism 414, for example, Velcro, on the ends of the cuff 404 to secure the cuff 404 in place when disposed around the arm of the subject. In some embodiments, the remote phlebotomy device 402 may also include a device stabilization mechanism (not shown) to stabilize the device 402 on the arm of the subject. For example, the cuff 404 may incorporate an inflatable portion or tourniquet-like mechanism. In some embodiments, the remote phlebotomy device 402 may also be configured to constrict around the arm of the subject to increase the diameter of the target vessel (e.g., a vein). For example, the remote phlebotomy device 402 may be configured to constrict around the arm of the subject to increase the diameter of the target vessel distal to the remote phlebotomy device (e.g., a cuff 404) due to impedance of venous blood return.

The remote phlebotomy device 404 can include image sensors (e.g., an ultrasound transducer array 416), a blood sampling assembly 418, and an electrical and control interface 420. While the example remote phlebotomy device 402 shown in FIGS. 4A and 4B includes an ultrasound transducer array, it should be understood that in some embodiments, other image sensor and imaging technologies may be used in the remote phlebotomy device 402. The ultrasound transducer array 416 may be configured to be connected to and in communication with a portable ultrasound system (e.g., an image acquisition system 112 shown in FIG. 1). The signal acquired by the ultrasound transducer array 416 may be provided to the ultrasound system to, for example, generate images. In some embodiments, as discussed above, an image analysis module (e.g., a machine learning network) configured to perform image analysis on the acquired ultrasound images may be implemented on the ultrasound system or other computer system (e.g., computer system 110 shown in FIG. 1) coupled to the ultrasound system. In some embodiments, the imagen analysis module (e.g., a machine learning network) may be trained to analyze or interpret the image data (or images) to determine, for example, a target vessel location and characteristics (e.g., vessel centroid depth, diameter, location along the ultrasound array, etc.). Segmentation of the target vessel may be based on machine learning of morphological and spatial information in the images of the region of interest and the target vessel (e.g., ultrasound images). In some embodiments, a neural network may be trained to learn features at multiple spatial and temporal scales. Vessels of interest may be distinguished based on shape and/or appearance of the vessel wall, shape and/or appearance pf surrounding tissues, and the like. Characteristics such as vessel diameter, etc., may be used to determine if a vessel is appropriate for needle insertion. In some embodiments, an insertion point may be determined (e.g., using the image analysis module) based on the determined location of the target vessel and calculating a depth and a pathway for a needle of the remote vascular access device 402 from the surface of the subject to the target vessel. In addition, in some embodiments, an acquired image or images can be analyzed (e.g., using an image analysis module) to detect critical structures that a needle should avoid and to compute a pathway, for example, from a surface (e.g., skin) of the subject to the target vessel such that the needle avoids the critical structures and intersects with the target vessel. The location and characteristic information determined by the image analysis module (e.g., a machine learning network) may be provided to, for example, the robotic blood sampling assembly 418 (e.g., the electrical control).

The electrical and control interface 420 can be configured to control various operations of the blood sampling assembly 418. In some embodiments, the electrical and control interface 420 can be coupled to a controller 434 (e.g., controller 126 shown in FIG. 1). The blood sampling assembly 518 can include a needle 422, pre-labeled blood vial(s) 424, a blood detection system 426, a needle injection system 428, and a needle positioning system that can include a needle angle control 430 and a needle translation track 432. In some embodiments, the needle 422 may be a standard 21 or 23 gauge needle for blood sampling. In some embodiments, one or more blood vials 424 may be provided in the blood sampling assembly 418. In some embodiments, the blood sampling assembly 418 may include up to four built-in blood vials 424. The blood sampling assembly 418 may be configured to include an automated flow control to fill the one or more vials 424. Accordingly, more than one vial 424 may be filled with blood, just as blood is often collected in several vials in a medical phlebotomy laboratory. The needle injection system 428 may be configured to actuate or deploy the needle 422 in response to, for example, an input received from controller 434 (e.g., a subject or caregiver pushes a button on the controller 434) or a command signal received by the electrical and control interface 420. In some embodiments, as discussed above, a command signal may be received from, for example, a computer system at an expert site or from an image analysis module at the remote site.

In some embodiments, the robotic blood sampling assembly 418 may be configured to allow for adjustment of the positioning of the needle 422 in the remote vascular access device 402. In some embodiments, the needle angle control 430 may be configured to adjust an angle of the needle 422 relative to a surface of the subject. In some embodiments, the blood sampling assembly 418 may advantageously be configured to provide an additional degree of freedom for the needle 422 which can advantageously allow automatic fine tuning of the position of the needle 422 with respect to the target vessel and an appropriate insertion point. For example, in some embodiments, the needle translation track 432 may be configured to allow a translational position of the needle 422 to be automatically adjusted along the ultrasound array 416. The additional degree of freedom can act to slide (e.g., along the needle translation track 432) the needle 422 across the ultrasound array 416 (e.g., the long axis of the ultrasound transducer array 416) for a “fine-positioning” step prior to needle insertion. This feature can be advantageous by enabling a user (e.g., a subject or caregiver) with limited dexterity to use the remote vascular access device 402. As a result, the user only needs to position the remote phlebotomy device 402 such that the target vessel is within the field of view of the ultrasound transducer array 416 (e.g., within approximately 4 cm).

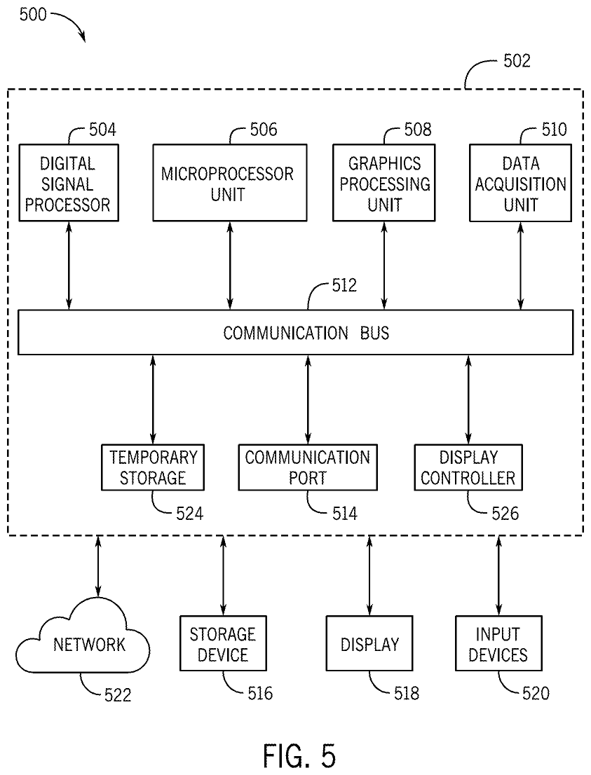

FIG. 5 is a block diagram of an example computer system in accordance with an embodiment. Computer system 500 may be used to implement various systems and methods described herein. In some embodiments, the computer system 500 may be a workstation, a notebook computer, a tablet device, a mobile device, a multimedia device, a network server, a mainframe, one or more controllers, one or more microcontrollers, or any other general-purpose or application-specific computing device. The computer system 500 may operate autonomously or semi-autonomously, or may read executable software instructions from the memory or storage device 516 or a computer-readable medium (e.g., a hard drive, a CD-ROM, flash memory), or may receive instructions via the input device 520 from a user, or any other source logically connected to a computer or device, such as another networked computer or server. Thus, in some embodiments, the computer system 500 can also include any suitable device for reading computer-readable storage media.

Data, such as data acquired with an imaging system (e.g., an ultrasound imaging system, optical imaging system, etc.) may be provided to the computer system 500 from a data storage device 516, and these data are received in a processing unit 502. In some embodiment, the processing unit 502 includes one or more processors. For example, the processing unit 502 may include one or more of a digital signal processor (DSP) 504, a microprocessor unit (MPU) 506, and a graphics processing unit (GPU) 508. The processing unit 502 also includes a data acquisition unit 510 that may be configured to electronically receive data to be processed. The DSP 504, MPU 506, GPU 508, and data acquisition unit 510 are all coupled to a communication bus 512. The communication bus 512 may be, for example, a group of wires, or a hardware used for switching data between the peripherals or between any component in the processing unit 502.

The processing unit 502 may also include a communication port 514 in electronic communication with other devices, which may include a storage device 516, a display 518, and one or more input devices 520. Examples of an input device 520 include, but are not limited to, a keyboard, a mouse, and a touch screen through which a user can provide an input. The storage device 516 may be configured to store data, which may include data such as image data, segmentation data, labeled images, whether these data are provided to, or processed by, the processing unit 502. The display 518 may be used to display images and other information, such as magnetic resonance images, patient health data, and so on.

The processing unit 502 can also be in electronic communication with a network 522 to transmit and receive data and other information. The communication port 514 can also be coupled to the processing unit 502 through a switched central resource, for example the communication bus 512. The processing unit can also include temporary storage 524 and a display controller 526. The temporary storage 524 can be configured to store temporary information. For example, the temporary storage 524 can be a random access memory.

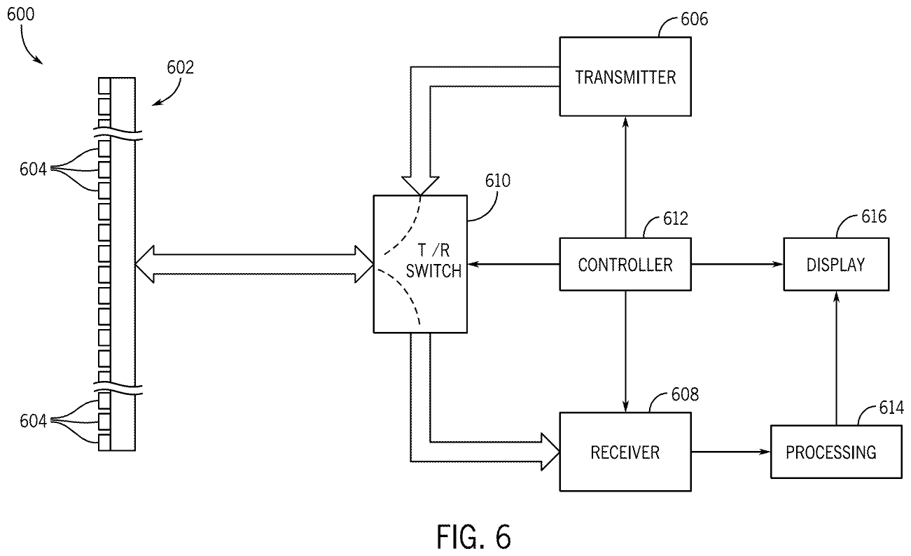

FIG. 6 is a schematic diagram of an example ultrasound system in accordance with an embodiment. FIG. 6 illustrates an example of an ultrasound system 600 that can be utilized to implement the systems and methods described in the present disclosure. The ultrasound system 600 includes a transducer array 602 that includes a plurality of separately driven transducer elements 604. The transducer array 602 can include any suitable ultrasound transducer array, including linear arrays, curved arrays, phased arrays, and so on. Similarly, the transducer array 602 can include a 1D transducer, a 1.5D transducer, a 1.75D transducer, a 2D transducer, a 3D transducer, and so on. As mentioned above, in some embodiments, a transducer array 604 may be incorporated into a remote vascular access device as shown in FIG. 4A and coupled to and in communication with, for example, a portable ultrasound system that can incorporate the remaining elements discussed below with respect to FIG. 6.

When energized by a transmitter 606, a given transducer element 604 produces a burst of ultrasonic energy. The ultrasonic energy reflected back to the transducer array 602 (e.g., an echo) from the object or subject under study can be converted to an electrical signal (e.g., an echo signal) by each transducer element 604 and can be applied separately to a receiver 608 through a set of switches 610. The transmitter 606, receiver 608, and switches 610 are operated under the control of a controller 612, which may include one or more processors. As one example, the controller 612 can include a computer system.

The transmitter 606 can be programmed to transmit unfocused or focused ultrasound waves. In some configurations, the transmitter 606 can also be programmed to transmit diverged waves, spherical waves, cylindrical waves, plane waves, or combinations thereof. Furthermore, the transmitter 606 can be programmed to transmit spatially or temporally encoded pulses.

In some configurations, the transmitter 606 and the receiver 608 can be programmed to implement a high frame rate. For instance, a frame rate associated with an acquisition pulse repetition frequency (“PRF”) of at least 100 Hz can be implemented. In some configurations, the ultrasound system 600 can sample and store at least one hundred ensembles of echo signals in the temporal direction.

The controller 612 can be programmed to implement an imaging sequence using the techniques described in the present disclosure, or as otherwise known in the art. In some embodiments, the controller 612 receives user inputs defining various factors used in the design of the imaging sequence.

A scan can be performed by setting the switches 610 to their transmit position, thereby directing the transmitter 606 to be turned on momentarily to energize transducer elements 604 during a single transmission event according to the implemented imaging sequence. The switches 610 can then be set to their receive position and the subsequent echo signals produced by the transducer elements 604 in response to one or more detected echoes are measured and applied to the receiver 608. The separate echo signals from the transducer elements 604 can be combined in the receiver 608 to produce a single echo signal.

The echo signals are communicated to a processing unit 614, which may be implemented by a hardware processor and memory, to process echo signals or images generated from echo signals. As an example, the processing unit 614 can generate images of a vessel of interest using the methods described in the present disclosure. Images produced from the echo signals by the processing unit 614 can be displayed on a display system 616.

Computer-executable instructions for supervised remote intervention according to the above-described methods may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access.

The present invention has been described in terms of one or more preferred embodiments, and it should be appreciated that many equivalents, alternatives, variations, and modifications, aside from those expressly stated, are possible and within the scope of the invention.

1. A method for remote intervention for a subject, the method comprising:
acquiring an image of a region of interest of the subject using an interventional device positioned on the subject and an image acquisition system, the region of interest including a target structure and the subject is located at a first site;
analyzing the acquired image using an image analysis module to identify and label the target structure in the region of interest;
transmitting the labelled image from the first site to a second site for expert review, wherein the second site is remote from the first site; and
receiving a command signal at the first site from the second site, the command signal generated based on the expert review of the labelled image and configured to control an action of the interventional device.

2. The method according to claim 1, wherein analyzing the acquired image further comprises analyzing the acquired image to detect critical structures that a needle should avoid and computing a pathway from a surface of the subject such that the needle avoids the critical structures and intersects with the target structure.

3. The method according to claim 1, further comprising causing the deployment of a needle of the interventional device based on the command signal.

4. The method according to claim 1, further comprising enabling the interventional device for deployment based on the command signal.

5. The method according to claim 3, further comprising causing the deployment of a needle of the interventional device.

6. The method according to claim 1, wherein the image analysis module is implemented as a machine learning network.

7. The method according to claim 1, wherein the interventional device is a vascular access device configured for drawing blood.

8. The method according to claim 1, wherein the interventional device is a vascular access device configured for intravenous medicine delivery.

9. The method according to claim 1, wherein the interventional device comprises an ultrasound transducer and the image acquisition system is an ultrasound system.

10. The method according to claim 1, wherein the interventional device comprises an optical image sensor and the image acquisition system is an optical imaging system.

11. The method according to claim 1, wherein transmitting the labelled image from the first site to a second site for expert review comprises transmitting the labelled image from the first site to the second site over a communication network.

12. The method according to claim 1, wherein the interventional device is a remote vascular access device, the target structure is a target vessel and analyzing the acquired image using an image analysis module to identify and label a target structure in the region of interest comprises determining one or more of a location of the target vessel, a centroid depth of the target vessel, and a diameter of the target vessel.

13. The method according to claim 12, further comprising determining if the target vessel is appropriate for needle insertion based on the determined diameter of the target vessel.

14. The method according to claim 1, wherein the interventional device is configured to be positioned around an arm of the subject.

15. The method according to claim 1, wherein the interventional device comprises a cuff configured to be positioned around an arm of the subject.

16. The method according to claim 1, wherein the command signal is further generated based on a user input received at the second site.

17. The method according to claim 1, further comprising monitoring the interventional device based on images acquired using the interventional device and the image acquisition system to determine a change in position of the interventional device.

18. A system for remote intervention for a subject, the system comprising:
an interventional device positioned on the subject, the interventional device comprising:
an image sensor;
a needle;
a robotic assembly comprising a needle positioning system configured to automatically adjust a position of the needle with respect to the image sensor to align the needle with a target structure in a region of interest of the subject; and
an image acquisition system coupled to the image sensor of the interventional device; and
an image analysis module coupled to the interventional device and the image acquisition system, the image analysis module configured to analyze an image of the region of interest of the subject to identify and label the target structure, wherein the image of the region of interest is acquired using the image sensor and image acquisition system.

19. The system according to claim 18, wherein the image analysis module is a machine learning network.

20. The system according to claim 18, wherein the needle positioning system is further configured to automatically adjust the position of the needle to align the needle with a target insertion point for the target structure and to avoid critical structures.

21. The system according to claim 18, wherein the interventional device is a vascular access device and further comprises a cuff configured to be positioned around an arm of the subject.

22. The system according to claim 18, wherein the interventional device is a vascular access device configured for drawing blood and further comprises one or more vials.

23. The system according to claim 18, wherein the interventional device is a vascular access device configured for intravenous medicine delivery.

24. The system according to claim 18, wherein the image sensor is a transducer array and the image acquisition system is an ultrasound system.

25. The system according to claim 18, wherein the image sensor is an optical image sensor and the image acquisition is an optical imaging system.

26. The system according to claim 18, wherein the interventional device is a vascular access device, the target structure is a target vessel, and the image analysis module is further configured to determine one or more of a location of the target vessel, a centroid depth of the target vessel, and a diameter of the target vessel.

27. The system according to claim 18, wherein the interventional device is a vascular access device configured to be positioned around an arm of the subject and to constrict around the arm of the subject to increase the diameter of the target vessel.

28. A method for remote intervention for a subject, the method comprising:
acquiring an image of a region of interest of the subject using an interventional device positioned on the subject and an image acquisition system, the region of interest including a target structure;
analyzing the acquired image using an image analysis module to identify and label the target structure in the region of interest; and
generating a command signal, using the image analysis module, the command signal generated based on the labelled image and configured to control an action of the interventional device.
